(19) United States
(12) Patent Application Publication
(10) Pub. No.: US 2015/0037253 A1
(43) Pub. Date: Feb. 5, 2015

(54) METAL COMPLEXES AS IMAGING AGENTS

(71) Applicant: The University of Melbourne, Parkville, Victoria (AU)

(72) Inventors: Paul Donnelly, Melbourne (AU); James Hickey, Melbourne (AU); Kevin Barnham, Melbourne (AU); Sin Chun Lim, Melbourne (AU)

(21) Appl. No.: 14/363,522

(22) PCT Filed: Dec. 6, 2012
(86) PCT No.: PCT/AU2012/001489

§ 371 Date: Jun. 6, 2014

(30) Foreign Application Priority Data
Dec. 6, 2011 (AU) 2011905075
Dec. 6, 2011 (AU) 2011905079

(51) Int. Cl. A61K 51/04; A61K 49/10; C07D 213/77; C07F 1/08; C07D 417/04

(52) U.S. Cl. CPC A61K 51/0472; C07F 1/08; C07D 417/04; C07D 213/77; A61K 49/10
USPC 424/1.65; 546/2; 546/270.1; 546/306; 424/9.361; 534/10

(57) ABSTRACT

The present invention relates to copper, gallium and technetium coordinated thiosemicarbazone-pyridylhydrazine (substituted at the pyridine ring with a substituted benzothiazole or stilbene moiety) complexes and methods thereof. Such compounds possess utility in PET imaging and diagnosis of amyloid diseases.

### FIELD

The present invention relates generally to chemical compounds and methods for their use and preparation. In particular, the invention relates to chemical compounds which may possess useful activity in the treatment, diagnosis and monitoring of, for instance, amyloid diseases in their early stages, and in particular, Alzheimer's disease.

### BACKGROUND

Amyloidosis is a general term that describes a number of diseases characterised by extracellular deposition of protein fibrils which form numerous ‘amyloid deposits’. These plaque-like deposits may occur in localised sites, such as the brain or systemically. The fibrillar composition of these deposits is an identifying characteristic for the various forms of amyloid disease. The following diseases and their associated protein have been identified as amyloid diseases: Diabetes mellitus type 2 (amylin); Alzheimer's disease (Aβ 39-42); Parkinson's disease (alpha-synuclein); Huntington's disease (huntingtin); Creutzfeldt-Jakob disease (PrP in cerebrum); congestive heart failure (PrP or transthyretin) and Bovine spongiform encephalopathy (PrP). Due to recent reports, Age related Macular Degeneration, ‘AMD’, is a further condition which may be characterized by amyloid deposits.

Alzheimer's disease (AD) is the most common cause of progressive dementia in the elderly population, AD is characterised by the presence of distinctive lesions in the patient's brain. These brain lesions include abnormal intracellular filaments called neurofibrillary tangles, and extracellular deposits of amyloid plaques. Amyloid deposits are also present in the walls of cerebral blood vessels of Alzheimer's patients. The major constituent of amyloid plaques has been identified as a 4 kilodalton peptide (39-43 residues) called beta-amyloid peptide (‘Abeta’ or ‘Aβ’). Alzheimer's disease brain tissue is characterised by Aβ plaques and observations suggest that Aβ deposition contributes to the destruction of neurons. Abeta has been shown to be toxic to mature neurons both in culture and in vivo.

Currently, there is no medication capable of curing or stopping the progression of any amyloid diseases, including AD. Therapies for AD such as inhibition of acetylcholinesterase (AchE)² activity and antagonism of N-methyl-D-aspatarte (NMDA) receptors produce only modest symptomatic improvements in some patients. Other therapeutic approaches currently in clinical development aim to control the levels of Aβ amyloid in the brain, either by immunization or through pharmacological manipulation. Drugs that target BACE and γ-secretase, the two enzymes responsible for Aβ production have concern due to side-effects of secretase inhibition since these enzymes are not specific and process a variety of substrates including the NOTCH protein.

The cure or disruption of amyloid diseases, particularly Alzheimer's disease, is further withheld by a lack of accurate and usable imaging and patient diagnostic techniques. For example, whilst data emerging from a range of ¹¹C—PIB studies demonstrates quantitative determination of brain Aβ non-invasively, therefore allowing monitoring of potential anti-amyloid therapeutic agents, the very short half-life of ¹¹C (˜20.4 min), precludes widespread application of ¹¹C—PIB in a relevant fashion in clinical settings. With a half life of 109.7 mins, ¹⁸F is also somewhat restrictive. ¹¹C—PIB also requires an in situ cyclotron (cost ˜$2M) for the production of the radio-isotope ¹¹C and both ¹¹C and ¹⁸F must be covalently attached to a molecule which can be synthetically challenging.

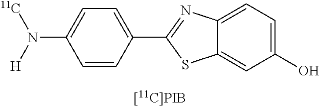

Accordingly, as well as providing therapeutics for treating amyloid diseases there is also a need for new imaging agents that target the underlying pathogenic mechanisms in amyloidosis type diseases, particularly AD, for early diagnosis of such disease states.

The present inventors have developed novel metal complexes that specifically bind to Aβ plaques for non-invasive diagnosis and monitoring of amyloid diseases in its early stages, before significant neuronal damage occurs.

### SUMMARY OF THE INVENTION

In one aspect the invention provides metal complexes of formula (I) or salts thereof:

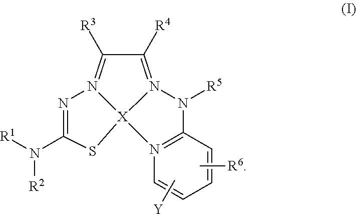

wherein:

X is Cu, Ga or Tc═O

Y is

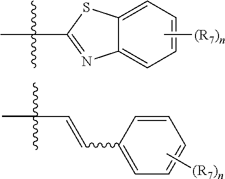

R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;

R³ and R⁴ are independently selected from hydrogen or C₁-C₁ alkyl, or R³ and R⁴ together form an optionally substituted aryl or optionally substituted cycloalkyl group;

R⁵ is selected from hydrogen or C₁-C₄ alkyl;

R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy, or amino;

R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy, or amino; and

n is 0-4.

The present invention also provides metal complexes of formula (Ia) or formula (Ib) or salts thereof:

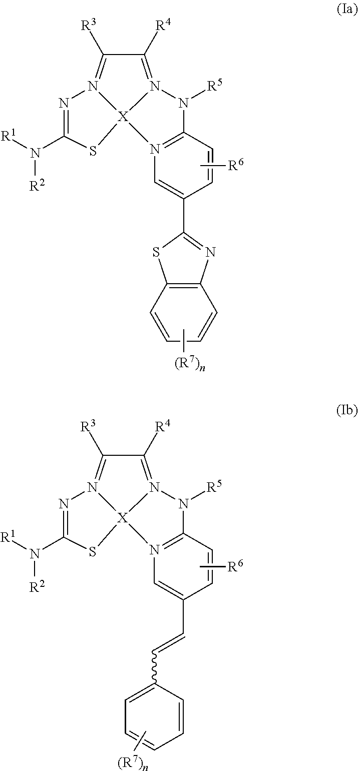

wherein:

X is Cu, Ga or Tc═O;

R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;

R³ and R⁴ are independently selected from hydrogen or C₁-C₄ alkyl, or R³ and R⁴ together form an optionally substituted aryl or optionally substituted cycloalkyl group;

R⁵ is selected from hydrogen or C₁-C₄ alkyl;

R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy, or amino;

R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy, or amino; and

n is 0-4.

A method of diagnosing an amyloid disorder comprising:

(i) administering a detectable quantity of a complex of formula (I), (Ia) or (Ib) or a salt thereof to a patient; and
(ii) detecting the binding of the complex to an amyloid deposit in said patient.

### BRIEF DESCRIPTION OF FIGURES

FIG. 1 ORTEP (40% probability) representation of the cation in dimer [Cu^{II}L¹]₂ 2BF₄.4DMF, solvent molecules and anions omitted for clarity.

FIG. 2 (a) UV/Vis and (b) Fluorescence spectra (λₑₓ=370 nm, λₑₘ=420 nm) of 1×10⁻⁵ M Cu^{II}L¹ in CH₃CN, (c) UV/Vis and (d) Fluorescence spectra (λₑₓ=390 nm, λ=480 nm) of 10 μM M Cu^{II}L² in CH₃CN.

FIG. 3 ORTEP (40% probability) representation of cationic helical dimer [Cu^{I}L²]²⁺, solvent molecules and anions omitted for clarity.

FIG. 4 ORTEP (40% probability) representation of cation [Cu^{II}L²]¹, solvent molecules and anion omitted for clarity.

FIG. 5a Cu^{II}L²b) AD human brain sections with 1E8 antibody stained Aβ plaques ×20 magnification; and c) epi-fluorescence of Cu^{II}L² binding selectively to Aβ plaques ×20 magnification image measured at λₑₓ=420 nm, λₑₘ=470 nm.

FIG. 5b Cu^{II}L³b) AD human brain sections with 1E8 antibody stained Aβ plaques ×20 magnification; and c) epi-fluorescence of Cu^{II}L³ binding selectively to Aβ plaques ×20 magnification, collated images measured at λₑₓ=359 nm, λₑₘ=461 nm; λₑₓ=420 nm, λₑₘ=470 nm; and λₑₓ=430 nm, λₑₘ=476 nm; overlaid.

FIG. 5c L³b) AD human brain sections with 1E8 antibody stained Aβ plaques ×20 magnification; and c) epi-fluorescence of L³ binding selectively to Aβ plaques ×20 magnification, collated images measured at λₑₓ=359 nm, λₑₘ=461 nm; λₑₓ=420 nm, λₑₘ=470 nm; and λₑₓ=430 nm, λₑₘ=476 nm; overlaid.

FIG. 6 Ligands for Log D partition coefficient comparison with ⁶⁴Cu complexes of the present invention.

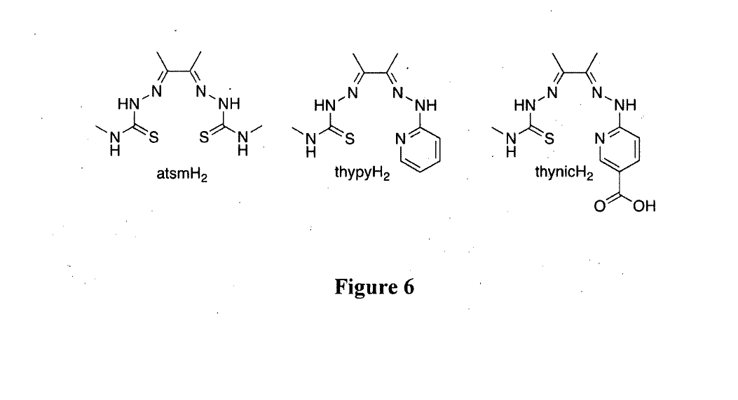

FIG. 7 a) Radio-HPLC of ⁶⁴Cu^{II}L² compared with ‘cold’ Cu^{II}L² (UV detection at 280 nm) and b) 3D collated biodistribution of ⁶⁴Cu^{II}L² in a Balb/c mouse showing direct accumulation of the radiotracer in both the lungs and liver. c) Radio-HPLC of ⁶⁴Cu^{II}L³ compared with ‘cold’ Cu^{II}L³ (UV detection at 280 nm) and d) 3D collated biodistribution of ⁶⁴Cu^{II}L³ in a Balb/c mouse showing improved accumulation of the radiotracer in the brain.

FIG. 8(a) Change in the UV/Vis spectrum of a solution containing H₂L² (20 μM) in 30% dmso/PB (20 mM, pH 7.4) upon titration with Cu²+ (1 mM). (b) Change in the Fluorescencespectrum of a solution containing H₂L² (10 μM) in 30% dmso/PB (20 mM, pH 7.4) upon titration with Cu²+ (1 mM), and (c) UV/Vis spectrum of 10 μM Cu^{II}L² in 30% dmso/PB (20 mM, pH 7.4).

FIG. 9 Cyclic voltammogram of Cu^{II}L¹ and Cu^{II}L². Scan rate 0.1 Vs⁻¹. Potentials are quoted relative to a SCE.

### DETAILED DESCRIPTION OF THE INVENTION

The invention is based on the discovery that the complexes of the general formula (I), as described in the above Summary of the invention hind with the metal binding site of an amyloid protein, thereby altering the protein conformation and function. Such complexes have significant potential in the treatment of or diagnosis of, a variety of disorders characterised by amyloid formation, herein referred to as “amyloid disorders”, and in particular Alzheimer's disease (‘AD’) and related conditions.

“Alkyl” refers to monovalent alkyl groups which may be straight chained or branched and preferably have from 1 to 10 carbon atoms or more preferably 1 to 6 carbon atoms.

Examples of such alkyl groups include methyl, ethyl, n-propyl, iso-propyl, n-butyl, iso-butyl, n-hexyl, and the like.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Aryloxy” refers to the group aryl-O— wherein the aryl group is as described above.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyl” refers to a monovalent alkenyl group which may be straight chained or branched and preferably have from 2 to 10 carbon atoms and more preferably 2 to 6 carbon atoms and have at least 1 and preferably from 1-2, carbon to carbon, double bonds. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Acyl” refers to groups H—C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl are as described herein.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Cycloalkyl” as used herein refers to cyclic alkyl groups having a single cyclic ring or multiple condensed rings, preferably incorporating 3 to 8 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, and the like.

“Halo” or “halogen” refers to fluoro, chloro, bromo and judo.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkynyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroaryloxy, heterocyclyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-acylamino, mono- and di-heteroaryl amino, mono- and di-heterocyclyl amino, and unsymmetric di-substituted amines having different substituents selected from alkyl, aryl, heteroaryl and heterocyclyl, and the like.

In an embodiment the complex is a metal complex of formula (Ia).

In an embodiment the complex is a metal complex of formula (Ib).

In an embodiment R¹ is hydrogen.

In an embodiment R¹ is hydrogen and R² is an optionally substituted C₁-C₆ alkyl.

In an embodiment R¹ is hydrogen and R² is C₁-C₆, alkyl.

In an embodiment R¹ is hydrogen and R² is a substituted C₁-C₆ alkyl.

In an embodiment R¹ is hydrogen and R² is a terminally substituted C₁-C₆ alkyl.

In an embodiment R¹ is hydrogen and R² is a C₁-C₆ alkyl terminally substituted with a group selected from halogen, NH₂, C₁-C₃ dialkyl amino, C₁-C₃ monoalkyl amino, aryl, trihalomethyl, acyl, and N-containing heteroaryl or N-containing heterocyclyl (for example, morpholinyl, piperidinyl, pyridinyl, thiomorpholinyl, piperazinyl, pyrrolidinyl or pyrrolyl).

In an embodiment R² is a C₁-C₃ alkyl terminally substituted with C₁-C₃ dialkyl amino or C₁-C₃ monoalkyl amino, or a bioisostere thereof.

In an embodiment R¹ is hydrogen and R² is C₁-C₃ alkyl or di C₁-C₃ alkyl amino ethyl.

In an embodiment R¹ is hydrogen and R² is methyl or dimethylaminoethyl.

In an embodiment R³ and R⁴ are independently C₁-C₃ alkyl.

In an embodiment R³ and R⁴ are both methyl.

In an embodiment R³ and R⁴ together form a 5-8 membered cycloalkyl.

In an embodiment R¹ is hydrogen, and R², R⁴ are independently C₁-C₃ alkyl.

In an embodiment R¹ is hydrogen, R³ and R⁴ are independently C₁-C₃ alkyl and R² is dimethylaminoethyl or a bioisostere thereof.

In an embodiment R⁵ is hydrogen.

In an embodiment R¹ and R⁵ are hydrogen, and R²-R⁴ are independently C₁-C₃ alkyl.

In an embodiment R¹ and R⁵ are hydrogen, R³ and R⁴ are C₁-C₃ alkyl and R² is dimethylaminoethyl or a bioisostere thereof.

In an embodiment R⁶ is hydrogen.

In an embodiment R⁶ is hydrogen and n=0.

In an embodiment R⁶ is hydrogen and n=1.

In an embodiment R¹, R⁵, and R⁶ are hydrogen, R³-R⁴ are independently C₁-C₃ alkyl or together form an optionally substituted aryl or optionally substituted cycloalkyl group and n=0, or 1.

In an embodiment R¹, R⁵, and R⁶ are hydrogen, R³-R⁴ are independently C₁-C₃ alkyl or together form an optionally substituted aryl or optionally substituted cycloalkyl group, R² is a terminally substituted C₁-C₆ alkyl and n 0, or 1.

When present, the substituents for R⁷ in compounds of formula (I), (Ia) or (Ib) may be selected from:

substituted aryl group, preferably halophenyl, aminophenyl, carboxyphenyl, hydroxyphenyl, cyanophenyl, nitrophenyl, trihaloalkylphenyl, and alkylphenyl.
alkoxy group, preferably methoxy and ethoxy;
amino group, preferably N-methylamino, and N,N′-dimethylamino.

In an embodiment n is 1 and R⁷ is dimethylamino, or a bioisostere thereof.

In an embodiment n is 1 and R⁷ is dimethylamino, or a bioisostere thereof, and R² is dimethylaminoethyl or a bioisostere thereof.

In an embodiment R¹, R⁵, and R⁶ are hydrogen, R³-R⁴ are independently C₁-C₃ alkyl or together form an optionally substituted aryl or optionally substituted cycloalkyl group R² is a terminally substituted C₁-C₆ alkyl, n=1 and R⁷ is dimethylamino.

In an embodiment X is Tc(═O), and preferably Tc-99m(═O).

In an embodiment X is Ga, and preferably ⁶⁸Ga.

In an embodiment X is Cu, preferably a positron-emitting isotope of Cu, for instance, ⁶⁰Cu, ⁶¹Cu, ⁶²Cu, or ⁶⁴Cu.

In an embodiment X is ⁶⁴Cu.

In an embodiment and with reference to formula (Ib) and formula (IIIb) below, the ligand system is the (E)-isomer.

The metal complexes of the present invention may be produced by complexing the tetradenate ligands of formula (IIa) or (IIb) with a stabilised copper reagent complex such as, for instance, Cu(OAc)₂, or a oxoTc(V) reagent complex

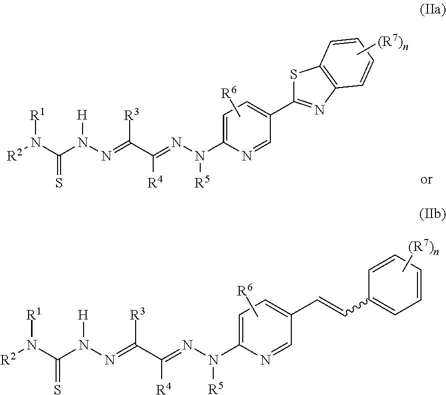

wherein R¹-R⁷ and n are as defined above.

This is preferably achieved by an exchange reaction between the ligand of formula (IIa) or formula (IIb) and a stabilising Cu(II), Ga(II) or oxoTc(V) reagent complex wherein the bond between the metal and stabilising complex is more labile than the bond that is formed between the transitional metal and ligand of formula (IIa) or (IIb). An example of a suitable stabilised reagent Cu(II) complex is Cu(OAc)₂. Generally, the stabilised metal complex will be dissolved in a suitable solvent followed by the addition of the ligand of formula (IIa) or (IIb). The addition of the ligand can be done either directly as a solid or as a solution in a suitable solvent which may or may not be the same solvent used to dissolve the transition metal complex. In the case where the solvents differ, the solvents are matched so as to avoid precipitation of the reactants from the reaction solvent mixture. Preferred solvents include polar solvents like alcohols, dimethylformamide, or chlorinated solvents like dichloromethane, chloroform, and carbontetrachloride, or aromatic hydrocarbons like benzene and toluene, or ethers like diethylether and tertrahydrofuran. The formation of the transition metal complex can usually be followed by observing colour changes in the reaction mixture or through spectroscopic means, such as for instance, G.C, UV/VIS spectrometry, or ESMS. The metal complexes of the present invention can be recovered by simply removing the reaction solvent in vacuo. The complex may be subjected to further purification according to known techniques or used without additional purification.

As a non-limiting example, the Cu(II) metal complexes of the present invention may be prepared according to scheme 1 and scheme 2 below:

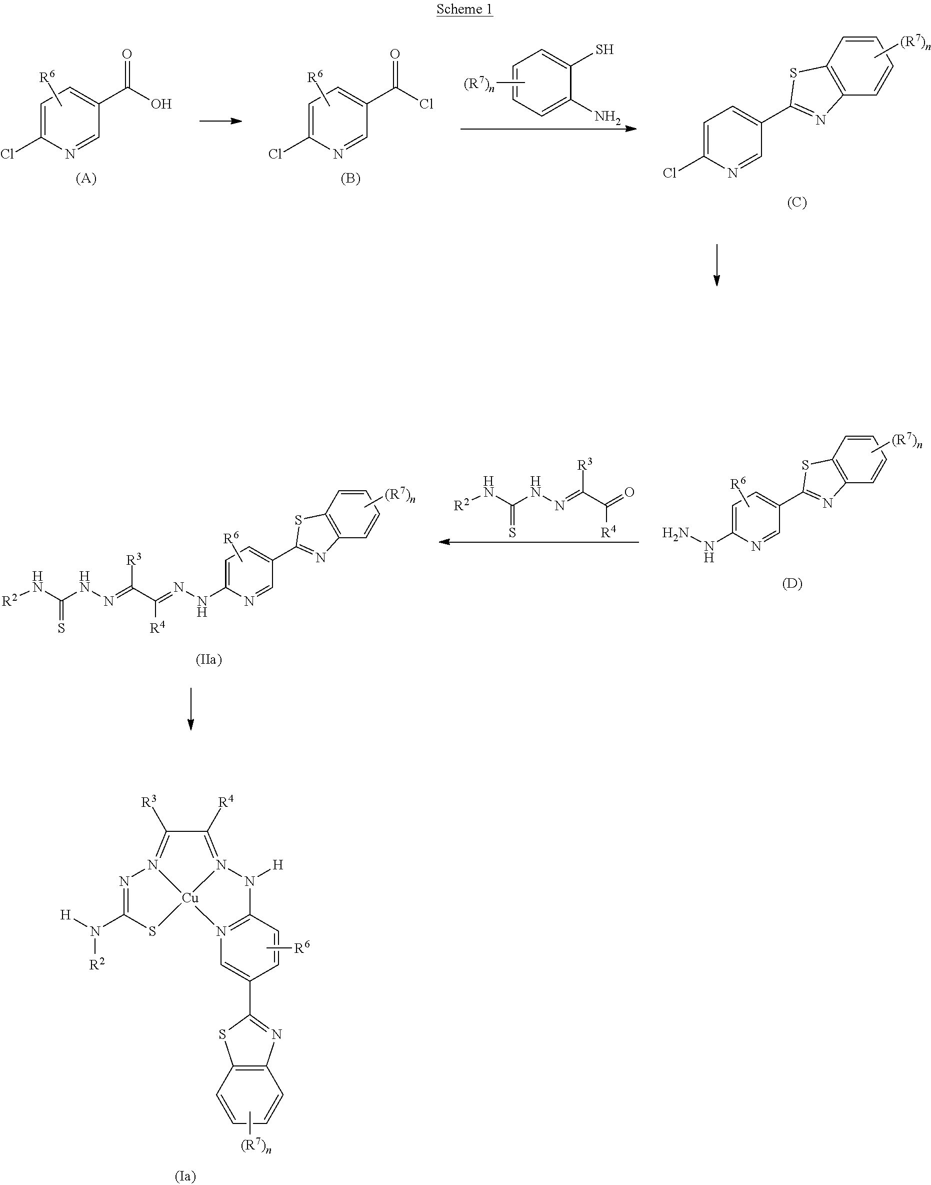

According to general Scheme 1 the acid chloride (B) of 2-chloronicotinic acid may be prepared by reacting with neat thionylchloride. Condensation of the acid chloride with a 2-aminothiopenol may afford (C). Aromatic substituted of the chloride with hydrazine affords (D). The hydrazine may be reacted with the shown thiosemicarbazone to prepare ligand (IIa). Complexation of (IIa) with a Cu(II) reagent complex such as Cu(II)(OAc)₂ may afford (Ia). This complexation, may be followed by ESMS and/or NMR. The oxoTc(V) complexes may be prepared in a similar fashion.

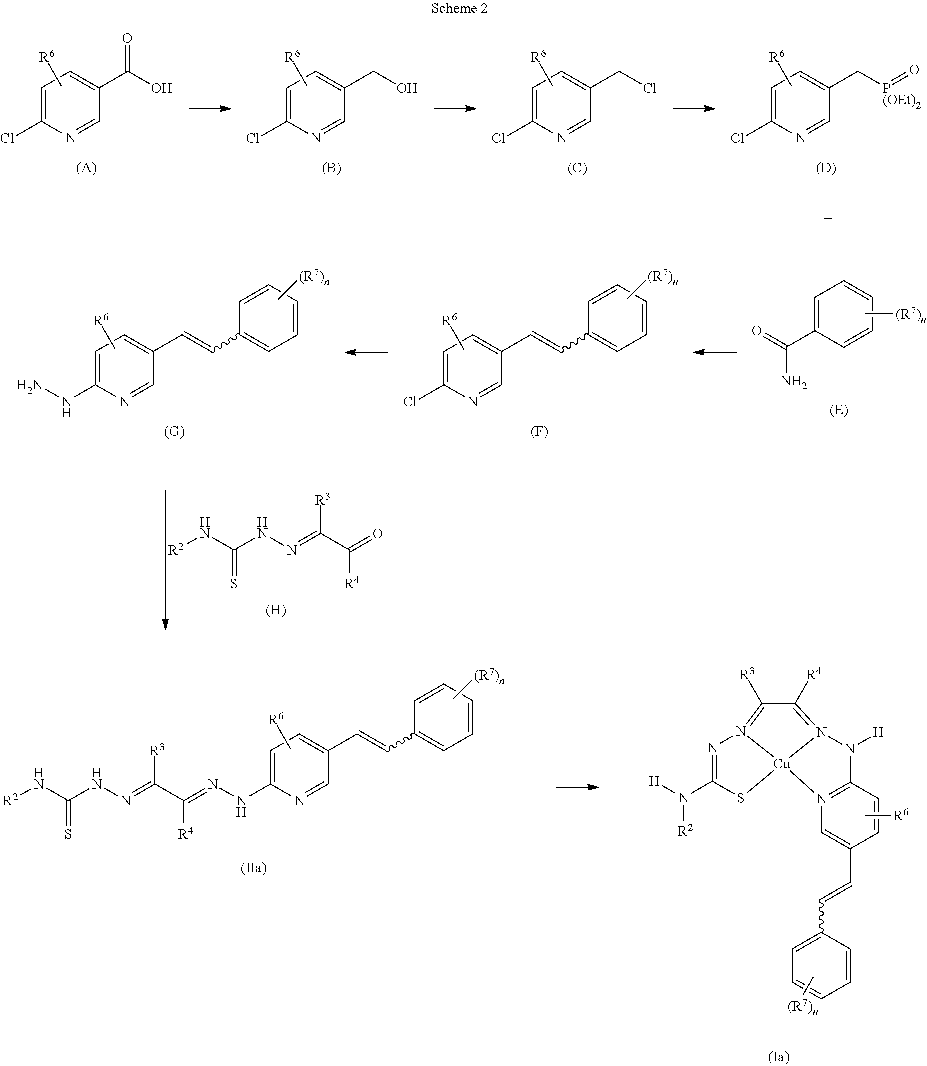

According to general Scheme 2 the compounds of the present invention may be prepared by reduction of the acid (A) to the alcohol (B), for instance with LiAlH₄. Chlorination of the alcohol (B) to (C) may be achieved with excess thionyl chloride under standard conditions. Treatment of the chloride with P(Oalkyl)₃ under Arbuzov rearrangement conditions affords the phosphonate (D) which can be coupled to (E) via a Horner-Wadsworth reaction to afford (F). Treatment of (F) with hydrazine hydrate and condensation with (H) affords (IIa). Complexation of (IIa) with a Cu(II) reagent complex such as Cu(II)(OAc)₂ may afford (Ia). The complexation may be followed by ESMS and NMR, as for instance, the geometrical isomer ratio of products from the Horner-Wadsworth reaction can be determined by ¹HNMR. The oxoTc(V) complexes may be prepared in a similar fashion.

During the reactions described above a number of the moieties may need to be protected. Suitable protecting groups are well known in industry and have been described in many references such as Protecting Groups in Organic Synthesis, Greene T W, Wiley-Interscience, New York, 1981.

Other compounds of formulae (I), (Ia) or (Ib) can be prepared by the addition, removal or modification of existing substituents. This could be achieved by using standard techniques for functional group inter-conversion that are well known in the industry, such as those described in “Comprehensive organic transformations: a guide to functional group preparations” by Larock R. C., New York, VCH Publishers, Inc. 1989.

Examples of functional group inter-conversions are: —C(O)NR*R** from —CO₂CH₃ by heating with or without catalytic metal cyanide, e.g. NaCN, and HNR*R** in CH₃OH; —OC(O)R from —OH with e.g., ClC(O)R in pyridine; —NC(S)NR*R** from —NHR with an alkylisothiocyanate or thiocyanic acid; —NRC(O)OR* from —NHR with alkyl chloroformate; —NRC(O)NR*R** from —NHR by treatment with an isocyanate, e.g. HN═C═O or RN═C═O; —NRC(O)R* from —NHR by treatment with ClC(O)R* in pyridine; —C(═NR)NR*R** from —C(NR*R**)SR with H₃NR^{r}OAe⁻ by heating in alcohol; —C(NR*R**)SR from —C(S)NR*R** with R—I in an inert solvent, e.g. acetone; —C(S)NR*R** (where R* or R** is not hydrogen) from —C(S)NH₂ with HNR*R**; C(═NCN)—NR*R** from —C(═NR*R**)—SR with NH₂CN by heating in anhydrous alcohol, alternatively from —C(═NH)—NR*R** by treatment with BrCN and NaOEt in EtOH; —NR—C(═NCN)SR from —NHR* by treatment with (RS)₂C═NCN; —NR**SO₂R from —NHR* by treatment with ClSO₂R by heating in pyridine; —NR*C(S)R from NR*C(O)R by treatment with Lawesson's reagent [2,4-bis(4-methoxyphenyl)-1,3,2,4-dithiadiphosphetane-2,4-disulfide]; —NRSO₂CF₃ from —NHR with triflic anhydride and base, —CH(NH₂)CHO from —CH(NH₂)C(O)OR* with Na(Hg) and HCl/EtOH; —CH₂C(O)OH from —C(O)OH by treatment with SOCl₂ then CH₂N₂ then H₂O/Ag₂O; —C(O)OH from —CH₂C(O)OCH₃ by treatment with PhMgX/HX then acetic anhydride then CrO₃; R—OC(O)R* from RC(O)R* by R**CO₃H; —CCH₂OH from —C(O)OR* with Na/R*OH; —CHCH₂ from CH₂CH₂OH by the Chugacv reaction; —NH₂ from —C(O)OH by the Curtius reaction; —NH₂ from —C(O)NHOH with TsCl/base then H₂O; —CHC(O)CHR from —CHCHONCHR by using the Dess-Martin Periodinane regent or CrO₃/aqII₂SO₄/acetone; —C₆H₅CHO from —C₆H₅CH₃ with CrO₂Cl₂; —CHO from —CN with SnCl₂/HCl; —CN from —C(O)NHR with PCl₅; —CH₂R from —C(O)R with N₂H₄/KOH.

From the above schemes it can be observed that compounds of formula (Ia) or (Ib) are key intermediates in the preparation of the metal complexes of the present invention.

Accordingly, in another aspect the invention provides novel compounds of formula (IIa) or a salt thereof:

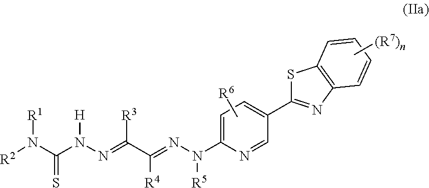

wherein

R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;

R³ and R⁴ are independently selected from hydrogen or C₁-C₄ alkyl, or R³ and R⁴ together form an optionally substituted aryl or optionally substituted cycloalkyl group;

R⁵ is selected from hydrogen or C₁-C₄ alkyl;

R⁶ is selected from hydrogen, hydroxy, halogen, carboxy, optionally substituted C₁-C₄ alkyl, optionally substituted C₁-C₄ alkoxy, optionally substituted aryl, optionally substituted aryloxy, or amino;

R⁷ at each occurrence is independently selected from hydroxy, halogen, carboxy, optionally substituted C₁-C₄ alkyl, optionally substituted C₁-C₄ alkoxy, optionally substituted aryl, optionally substituted aryloxy, or amino; and n is 0-4.

Accordingly, in another aspect the invention provides novel compounds of formula (IIb) or salts thereof:

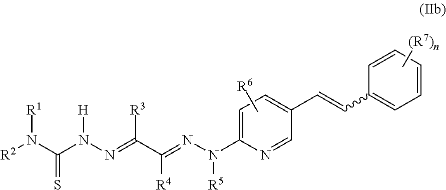

wherein:

R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;

R³ and R⁴ are independently selected from hydrogen or C₁-C₄ alkyl, or R³ and R⁴ together form an optionally substituted aryl or optionally substituted cycloalkyl group;

R⁵ is selected from hydrogen or C₁-C₄ alkyl;

R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy or amino;

R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy or amino; and

n is 0-4.

Without wishing to be bound by theory it is believed that the metal complexes of the present invention act by binding to amyloid proteins which form Aβ plaques. In particular it is postulated that the metal complexes of the invention with appended stilbene and benzothiazole moieties selectively binds to Aβ plaques. A particular advantage is that the ligands of the metal complexes of the present invention show selectivity for amyloid plaques over other β-select aggregates such as neurofibrillary tangles and Lewy bodies. This suggests that the ligands and thus the complexes have potential to be used in differential diagnosis of AD from other conditions. In addition to this as copper-64 is a positron emitter with a half-life of 12.7 hours, the complexes of the present invention are therefore well suited for PET imaging of Aβ plaques when the Cu(II) isotope is ⁶⁴Cu. As a further advantage the new stilbene based ligands form stable Cu(II) complexes which are more resistant to physiological reduction compared to similar types of Cu(II) systems. Such prior art systems for examples (see scheme 3 below), are only typically reduced to Cu(I) in hypoxic cells and are therefore being investigated as a hypoxia imaging agent in cancer research.

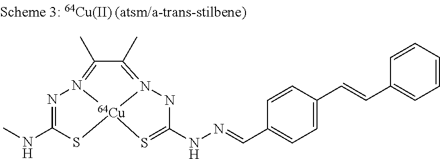

The compounds of the invention also show better drug-likeness and in a preferred embodiment are able to cross the blood brain barrier in biodistribution studies.

In an embodiment the Cu(II) complexes of the present invention are postulated to be paramagnetic contrast agents and therefore may function to amplify the magnetic resonance (MR) properties of water. In turn such contrast agents may find specific utility in magnetic resonance imaging (MRI) techniques.

Thus, the compounds of the present invention may be used in diagnosis and monitoring a variety of amyloid forming disorders. Such disorders include diabetes mellitus type 2, Alzheimer's disease (AD), Parkinson's disease, Huntington's disease, Creutzfeldt-Jakob disease, congestive heart failure, bovine spongiform encephalopathy and age related Macular Degeneration (AMD).

The invention also provides for the use of a compound of formula (I), (Ia) or (Ib) in the manufacture of a medicament for diagnosis and monitoring an amyloid disorder.

Preferably, the metal complexes of the present invention may be administered to a subject as a pharmaceutically acceptable salt. It will be appreciated however that non-pharmaceutically acceptable salts also fall within the scope of the present invention since these may be useful as intermediates in the preparation of pharmaceutically acceptable salts or in veterinary applications. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of a metal complexe of formula (I), (Ia) or (Ib) is also within the scope and spirit of the invention. The term “pro-drug” is used in its broadest sense and encompasses those derivatives that are converted in vivo to the compounds of the invention. Such derivatives would readily occur to those skilled in the art, and include, for example, compounds where a free hydroxy group (for instance at the R⁷ position) is converted into an ester, such as an acetate or phosphate ester, or where a free amino group is (for instance at the R⁷ position) converted into an amide (eg. α-aminoacid amide). Procedures for esterifying, eg. acylating, the compounds of the invention are well known in the art and may include treatment of the compound with an appropriate carboxylic acid, anhydride or chloride in the presence of a suitable catalyst or base.

The complexes of the invention may be in crystalline form either as the free compounds or as solvates (e.g. hydrates) and it is intended that both forms are within the scope of the present invention. Methods of salvation are generally known within the art.

As stated earlier the metal complexes of the present invention are useful as diagnostic tools.

For instance, complexes may be useful in amyloid imaging techniques for diagnosing and/or monitoring amyloid diseases in vivo (i.e., antemortem). Such complexes may also be useful in quantitation of amyloid deposits in biopsy or post-mortem tissue specimens.

In a further embodiment the invention provides a method of diagnosing an amyloid disorder comprising:
(i) administering a detectable quantity of a complex of formula (I), (Ia) or (Ib) or a salt thereof to a patient, and
(ii) detecting the binding of the complex to an amyloid deposit in said patient.

The method described above may be used to diagnose a patient who is suspected of having an amyloidosis associated disease. The method can also be used to determine the presence, size and location of amyloid deposits in the body (preferably the brain) of the patient.

The diagnostic methods disclosed herein refer to the use of the transition metal complexes of the present invention in conjunction with non-invasive imaging techniques such as magnetic resonance spectroscopy (MRS), magnetic resonance imaging (MRI), gamma imaging such as positron emission tomography (PET) or single-photon emission computed tomography (SPECT). In an embodiment where the Cu(II) isotope is ⁶⁴Cu, preferably the imaging technique is PET. Such techniques can be used to quantify and diagnose amyloid depositions in vivo.

The amount of administered transition metal complex to be used in the diagnosis method will depend on the age, sex, weight and condition of the patient. This can be adjusted as required by a skilled physician. It will be appreciated by those in the art that the quantity of the labelled probe required for diagnostic imaging will be relatively minute. Dosages can range from 0.001 mg/kg to 1000 mg/kg, however smaller quantities in the range of 0.1 mg/kg to 100 mg/kg will be preferred.

The attending diagnostic physician may administer the metal complex of the present invention either locally or systemically (for instance, intravenously, intrathecally, intraarterially, and so on). After administration the metal complex is allowed sufficient time to bind with an amyloid protein. This can take between 30 minutes to 2 days. The area of the patient under investigation is then scanned by the standard imaging techniques discussed above. In relation to brain imaging, for example AD diagnosis, preferably the amount of the bound metal complex (total and specific binding) is measured and compared as a ratio with the amount of metal complex bound to the cerebellum of the patient. This ratio is then compared to the same ratio in an age-matched normal brain.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

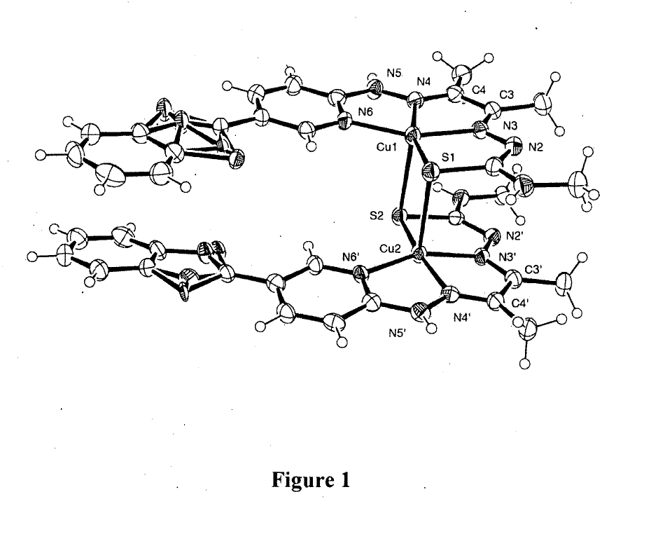

The monocationic tetrafluoroborate salt [Cu^{II}(HL¹)]BF₄ was isolated as purple crystals suitable for single crystal X-ray studies from the oxidation and deprotonation of the [Cu^{I}(H₂L¹)]BF₄ complex in a mixture of dimethylformamide and diethylether (FIG. 1). The copper is in an expected four coordinate 5-5-5 (N,N,N,S) chelate ring distorted square planar geometry. An axial association to a sulfur of an adjacent molecule [CuI—S2=2.803(2) Å] and π-π stacking (ca. 3.7 Å) between the benzothiazole rings results in the formation of a dimer and a tendency towards square pyramidal geometry with the copper 0.165(×) Å out of the plane. The N3-CuI—S1 bond angle (85.76(13)°) is significantly larger than the bond angle for N4-CuI—N6(80.70(16)°) and deprotonation of the thiosemicarbazonato limb is reflected in the C2-S1 distance of 1.773(4) Å and the C2-N2 distance of 1.333(6) Å, consistent with previously reported Cu^{II}THYNIC.⁽¹⁾

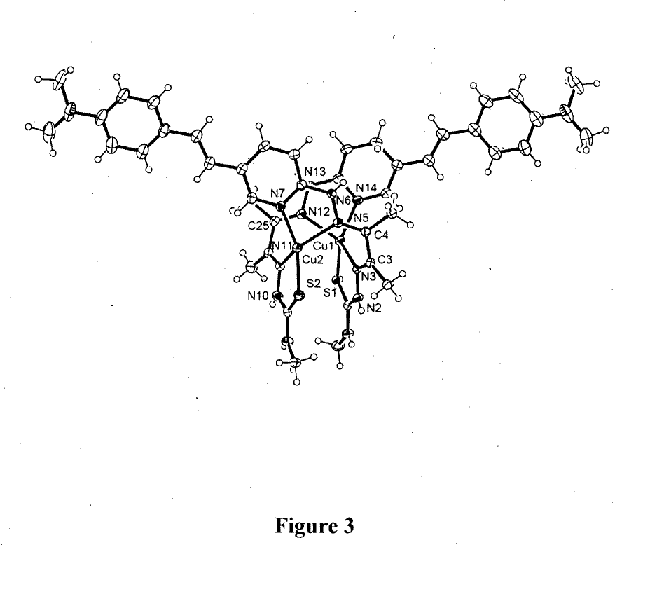

The reaction of H₂L² with [Cu^{I}(CH₃CN)₄]PF₆ in dimethylformamide followed by addition of diethlyether resulted in the precipitation of red crystals of a Cu^{I} complex. [Cu^{I}(H₂L²)]BF₄ identified by single crystal X-ray crystallography. An ORTEP representation of the dimeric cation in FIG. 3 displays the elegant helical coordination of two ligands bridging two copper atoms, with the ligand binding in a bidentate N—S to one Cu^{I} and N—N_{py} to the other. Significant torsion about the C—C ligand backbone (N3-C3-C4-N5=46.6(3)° and N11-C24-C25-N12=48.6(3)°) permits a distorted tetrahedral geometry for each Cu^{I} ion, with the distance between copper atoms Cu—Cu (3.398(×) Å) suggesting little interaction. The ligand remains protonated, as suggested by the ‘thione-like’ bond lengths of 1.693(2) Å for C2-S1 and 1.698(2) Å for C23-S2, with the structure analogous to Cu^{I}THYNIC and Cu^{I}atsm.^{(1,2)}

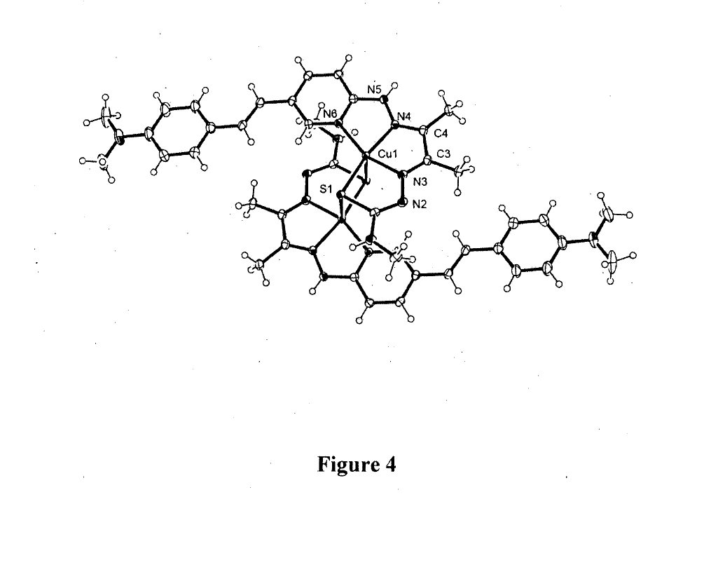

The Cu^{I} complex prepared from H₂L², [Cu^{I}(H₂L²)]BF₄ is stable when crystalline but readily oxidises in solution in the presence of air to give [Cu^{II}(HL²)]BF₄. Dark blue crystals of [Cu^{II}(HL²)]BF₄ revealed a centrosymmetric-dimer contrasting non-centrosymmetric [Cu^{II}(HL¹)]BF₄. As detailed in the ORTEP representation of [Cu^{II}(HL²)]BF₄ in FIG. 4, the E-conformer of the stilbene is not only confirmed but clearly preferring to orient in an opposing direction. Reorganisation of the ligands about the metal atoms in [Cu^{I}(H₂L²)]BF₄ results in a distorted square planar 5-5-5 (N,N,N,S) chelate ring.

### Electrochemistry and Electronic Spectroscopy of Cu^{II}L¹ and Cu^{II}L²

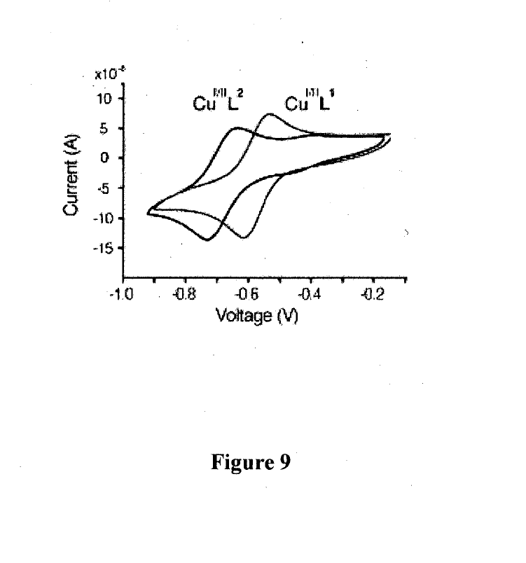

Cyclic voltammetry measurements of Cu^{II}btsc complexes in dimethyl formamide (DMF) or dimethylsulfoxide (DMSO) have proved useful indicators in predicting the in vivo dissociation of the complex, with clear correlation between reduction potential and likely intracellular reduction. The hypoxia selectivity of ⁶⁴Cu^{II}atsm is thought to be a consequence of the neutral complex diffusing into all cells but only being trapped in hypoxic cells by virtue of reduction of the Cu^{II} to Cu^{I}. Cyclic voltammetry measurements in DMF of the neutral complexes Cu^{II}L^{I} and Cu^{II}L² show both complexes undergo quasi-reversible reduction processes at a glassy carbon electrode tentatively attributed to a Cu^{II}/Cu^{I} couple, although it is acknowledged that DFT on the closely related Cu^{II}(atsm) suggested that in that case the LUMO does have some ligand character. The electron donating N,N-dimethylaminostilbene functional group present in Cu^{II}L² results in a lower reduction potential of −0.68 V vs. SCE ((ΔE=0.09 V, I_{c}/Iₐ=4.03), when compared to the benzothiazole functionalized compound in Cu^{II}L¹, Eₘ=−0.58 V versus SCE (ΔE=0.07 V, I_{c}/Iₐ=1.07) (FIG. 9). Under the same conditions Fc/Fc⁺:Em=0.53 V (ΔE=0.158 V, I_{c}/Iₐ=1.29) and CuII (atsm) Em=−0.59 V (ΔE=0.08 V, I_{c}/Iₐ=0.89). Given that Cu^{II}(atsm) is sufficiently stable for imaging applications the measured reduction potentials suggest that Cu^{II}L¹ and Cu^{II}L² will be sufficiently resistant to reductively assisted loss of the metal ion from the chelate encountered in most cellular environments.

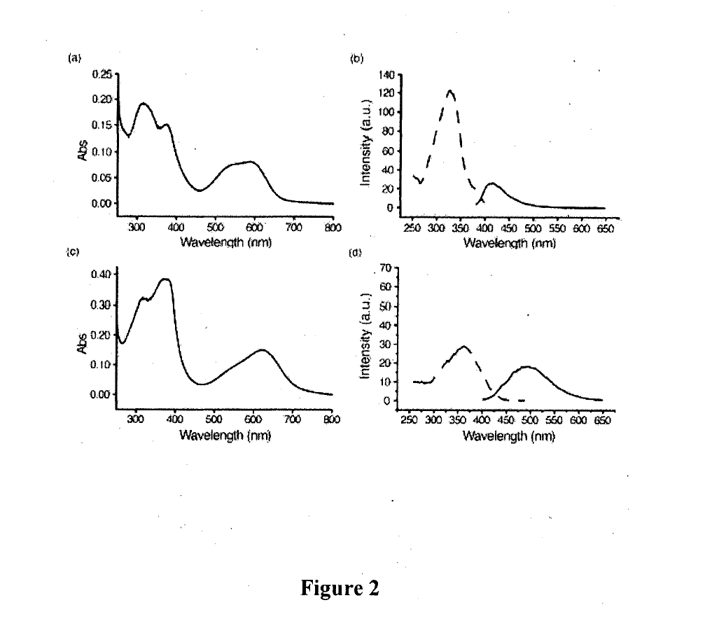

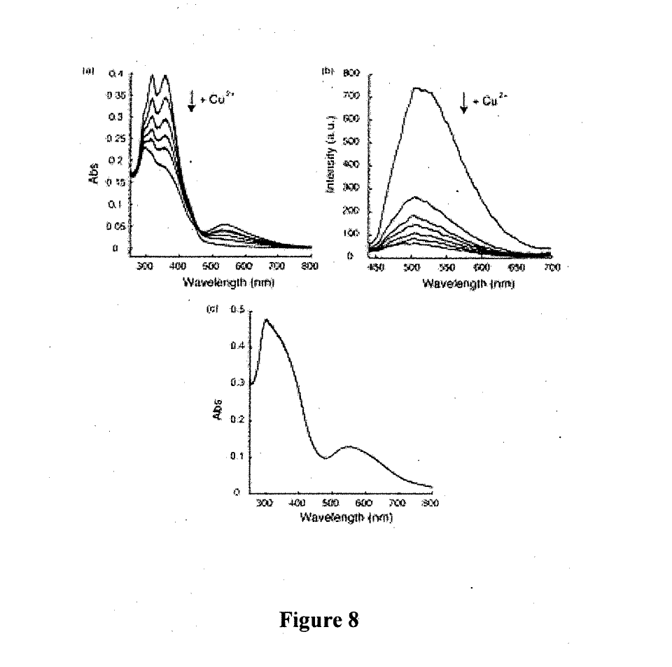

The complexes Cu^{II}L¹ and Cu^{II}L² display similar electronic spectra, with ligand based absorbances centered at λ_{abs}=300 nm (Cu^{II}L¹, ε=1.8×10⁴ M and Cu^{II}L², ε=3.2×10⁴ M) and λ_{abs}=380 nm (Cu^{II}L¹, ε=1.5×10⁴ M and Cu^{II}L², ε=3.8×10⁴ M), along with a broad absorbance between λ_{abs}=500-650 nm (Cu^{II}L¹λ₅₈₀, ε=8×10³ M and Cu^{II}L²λ₆₂₀, ε=1.4×10⁴ M) characteristic of metal to ligand charge transfer (MLCT) transitions (FIG. 2). The characteristic MLCT and ligand-based absorbances were chosen to monitor the stability of the complex Cu^{II}L² by RP-HPLC in the presence of intracellular reducing agent glutathione (GSH). A degassed solution of Cu^{II}L² (1×10⁴ M) was incubated in the presence of 100-fold GSH (30% DMSO/PB 20 mM, pH 7.4) over 4 hours at 37° C., with aliquots analysed by RP-HPLC monitoring the select absorbances. No significant change was observed suggesting that Cu^{II}L² is sufficiently stable towards intracellular reductant GSH and suitable to provide a chelate for ⁶⁴Cu and labeling motif for extracellular Aβ plaques. Titration of Cu²⁺ into a solution of H₂L² at pH 7.4 (FIG. 8) elegantly displays the transition from the free ligand to the doubly deprotonated neutral coordination complex, Cu^{II}L². This suggests that the speciation of ⁶⁴ Cu^{II}L² under biological conditions should be neutral, rather than potentially positively charged due to the hydrazinic limb of the ligand remaining protonated, as seen in the crystallography.

The weakly fluorescent complexes both retain the native fluorescent properties of the highly fluorescent ligands H₂L¹ and H₂L², despite the coordination of Cu^{II} resulting in a significant quench (FIG. 2). The effect of Cu^{II} coordination on the fluorescence of H₂L² is evident in FIG. 8b. Cu^{II}L¹ displays a broad emission centered at λₑₘ=420 nm when excited at λₑₓ=370 nm, and in the case of Cu^{II}L² a significant stokes shift was observed with an emission of λₑₘ=480 nm when excited at λₑₓ=390 nm. The weak fluorescence is perfectly suited to investigate the binding interaction of the complexes with Aβ plaques in human brain tissue.

### General Procedures

### Experimental

Crystallography.

Crystals were mounted in low temperature oil then flash cooled to 130 K using an Oxford low temperature device. Intensity data were collected at 130 K with an Oxford XCalibur X-ray diffractometer with Sapphire CCD detector using Cu—Kα radiation (graphite crystal monochromator λ1.54184 Å). Data were reduced and corrected for absorption.(³⁾ The structures were solved by direct methods and, difference fourier synthesis using the SHELX suite of programs⁴ as implemented within the WINGX⁵ software. Thermal ellipsoid plots were generated using the program ORTEP-3⁶ integrated within the WINGX suite of programs.

### General Procedures

Syntheses.

All reagents and solvents were obtained from commercial sources (Sigma-Aldrich) and used as received unless otherwise stated. Diacetyl-mono-4-methyl-3-thiosemicarbazone was prepared according to previous reports.^{(7,8)} Elemental analyses for C, H, and N were carried out by Chemical & MicroAnalytical Services Pty. Ltd, Vic. NMR spectra were recorded on a Varian FT-NMR 500 spectrometer (¹H NMR at 499.9 MHz and ¹³C{¹H} NMR at 125.7 MHz) at 298 K and referenced to the internal solvent residue.⁹ Mass spectra were recorded on an Agilent 6510-Q-TOF LC/MS mass spectrometer and calibrated to internal references.

UV/Visible spectroscopy.

UV/Vis spectra were recorded on a Cary 300 Bio UV-Vis spectrophotometer, from 800-250 nm at 0.5 nm data intervals with a 600 nm/min scan rate.

Fluorescence Spectroscopy.

Fluorescence emission spectra were measured on a Varian Cary Eclipse Fluorescence spectrophotometer.

High Pressure Liquid Chromatography.

Analytical RP-HPLC traces were acquired using an Agilent 1200 series PIPLC system equipped with a Agilent Zorbax Eclipse XDB-C18 column (4.6×150 mm, 5 mm) with a 1 ml/min flow rate and UV spectroscopic detection at 214 nm, 220 nm, and 270 nm. Retention times (Rₜ/min) were recorded using a gradient elution method of 0-100% B over 25 min, solution A consisted of water (buffered with 0.1% trifluoroacetic acid) and solution B consisted of acetonitrile (buffered with 0.1% trifluoroacetic acid).

Electrochemistry.

Cyclic voltammograms were recorded using an AUTOLAB PGSTAT100 equipped with GPES V4.9 software. Measurements of the complexes were carried out at approximately 1×10⁻³ M in dimethylformamide with tetrabutylammonium tetrafluoroborate (1×10⁻¹ M) as electrolyte using a glassy carbon disk (d, 3 mm) working electrode, a Pt wire counter/auxiliary electrode, and a Ag/Ag⁺ pseudo reference electrode (silver wire in H₂O (KCl (0.1 M)) AgNO₃ (0.01 M)). Ferrocene was used as an internal reference (Eₘ(Fc/Fc⁺)=0.54 V vs. SCE), where Eₘ refers to the midpoint between a reversible reductive (E_{pc}) and oxidative (Eₚₐ) couple, given by Eₘ=(E_{pc}+Eₚₐ)/2. Irreversible systems are only given reductive (E_{pc}) and oxidative (Eₚₐ) values, respectively.

Fluorescence Staining of Human AD Brain. Tissues(¹⁰⁾

Paraffin preserved brain tissue blocks were provided by the Victoria Brain Bank Network. Brain tissue was collected at autopsy. The National Neural Tissue Resource Centre performed Sourcing and preparation of human brain tissue. AD pathologic diagnosis was made according to standard National Institute on Aging-Reagan Institute criteria. Determination of age-matched Human control (HC) cases was subject to the above criteria. The AD and HC brain tissues sections (7 μM) were first de-paraffined (xylene, 3×2 min) followed by rehydration (soaking in a series of 100%, 90%, 70% and 0% v/v ethanol/di water). The hydrated tissue sections were washed in phosphate buffer saline (PBS, 5 min). Auto-fluorescence of the tissue was quenched using potassium permanganate (0.25% in PBS, 20 min) and washing with PBS (2×2 min) to remove the excess. The now brown coloured sections were washed with potassium metabisulfite and oxalic acid (1% in PBS) until the brown colour was removed followed by washing with PBS (3×2 min). The sections were blocked with bovine serum albumin (2% BSA in PBS, pH 7.0, 10 min) and covered with filtered Cu^{II}(L) (200 μM in 10% v/v dmso/PBS, 30 min). The sections were treated with BSA again to remove any Cu^{II}(L) non-specifically bound to the tissue. Finally, the sections were washed with PBS (3×2 min), di water and mounted with non-fluorescent mounting media (Dako), Fluorescence images were visualised using a Leica (Bannockburn, Ill.) DM1RB microscope.

### Synthetic Protocols

### 2-Chloropyridinyl-4-benzothiazole

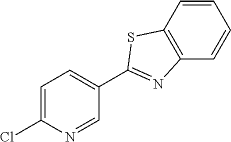 [SMILES: Clc1ccc(-c2nc3ccccc3s2)cn1]

2-Chloronicotinic acid (1.00 g, 6.35 mmol) was refluxed in thionylchloride (10 mL) under nitrogen for 1 hour. On cooling to room temperature volatiles were removed in vacuo. The residue was treated dropwise with a solution of 2-aminothiophenol (680 uL, 6.35 mmol) in THF (50 mL) over 10-15 minutes, and stirred at room temperature for a further hour. The reaction was diluted with CH₂Cl₂ (20 mL) and neutralised with sat. NaHCO₃ (50 mL). The organic layer was separated and the aqueous washed with dichloromethane (3×20 mL). Organics were combined, dried over MgSO₄, filtered, and volatiles were removed. The residue was subsequently chromatographed (CH₂Cl₂) to give a white solid (750 mg, 48%), ¹H NMR (500 MHz; DMSO-d₆): δ/ppm 9.07 (d, ⁴J_{HH}=2.6, 1H, PyH), 8.46 (dd, ³J_{HH}=8.3, ⁴J_{HH}=2.6, 1H, PyH), 8.18 (dt, ³J_{HH}=8, ⁴J_{HH}=0.6, 1H, ArH), 8.09 (dt, ³J_{HH}=8.1, ⁴J_{HH}=0.5, 1H, ArH), 7.57 (d, ³J_{HH}=8.4, 1H, PyH), 7.57 (ddt, ³J_{HH}=8.2, ³J_{HH}=7.2, ⁴J_{HH}=1, 1H, ArH). 7.50 (ddt, ³J_{HH}=8.1, ³J_{HH}=7.1, ⁴J_{HH}=0.9, 1H, ArH). ¹³C{¹H} NMR (125.7 MHz; DMSO-d₆): δ/ppm 163.1 (BzC), 153.2 (ArC), 152.3 (PyC), 147.9 (PyCH), 137.9 (PyCH), 134.6 (ArC), 128.3 (PyCCl), 126.9 (ArCH), 126.0 (ArCH), 124.9 (PyCH), 123.1 (ArCH), 122.5 (ArCH).

### 2-Chloropyridinyl-4-methylenediethylphosphonate

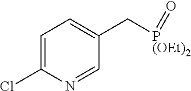

2-Chloronicotinic acid (3.00 g, 19.0 mmol) was dissolved in dry THF (60 mL) and cooled to 0° C. Lithium aluminiumhydride (870 mg, 23.0, mmol) was charged into the stirred reaction (CAUTION: gas evolution) followed by gradual warming to reflux for 4 hours. The reaction was quenched with sequential addition of wet THF (5 mL) and water (50 mL, cautiously) before filtration through celite and removal of volatiles in vacuo gave yellow oil that was purified by flash chromatography (SiO₂, CH₂Cl₂ followed by EtOAc). The crystalline alcohol was dissolved in CH₂Cl₂ (20 mL) and excess thionylchloride (5-10 mL) before refluxing for 1 hour. On cooling to room temperature, volatiles were removed in vacuo and the residue was neutralised with sat. NaHCO₃ before extraction with CH₂Cl₂ (3×40 mL). Organics were combined, dried over MgSO₄, filtered and concentrated to 3-5 mL before purification through a silica plug (eluting with CH₂Cl₂). Removal of volatiles in vacuo gave yellow oil as the desired 2-chloropyridyl-5-methylenechloride. The alkyl chloride was dissolved in triethylphosphite (10 mL) and heated to 140° C. for 2 hours. On cooling to room temperature, volatiles were removed in vacuo and the residue purified by flash chromatography (SiO₂, CH₂Cl₂ followed by EtOAc) to give a light yellow oil( ). ¹H NMR (500 MHz; CDCl₃): δ/ppm 8.28-8.26 (m, 1H, Py-H), 7.64 (dt, 1H, ³J_{HH}=8.2, ⁴J_{HH}=2.5, Py-H), 7.28 (d, 1H, ³J_{HH}=8.2, Py-H), 4.09-4.02 (m, 4H, O—CH₂), 3.09 (d, 2H, ³J_{HP}=21.6, O═P—CH₂), 1.28-1.25 (m, 6H, CH₃). ¹³C{¹H} NMR (125.7 MHz; CDCl₃); δ/ppm 150.4 (d, ³J_{CP}=7.7, PyCH), 150.3 (d, ⁵J_{CP}=4, PyC—Cl), 140.0 (d, ³J_{CP}=5.5, PyCH), 127.1 (d, ²J_{CP}=9, PyC), 124.2 (d, ⁴J_{CP}=2.9, PyCH). 62.6 (d, ²J_{CP}=6.8, O—CH₂), 30.5 (d, ¹J_{CP}=140.0, O═P—CH₂), 16.5 (d, ³J_{CP}=5.9, CH₂—CH₃). ³¹P {¹H} NMR (202.5 MHz; CDCl₃): δ/ppm 24.8 (s, O═P)

### (E)-2-Chloro-pyridinyl-4-(4′-N,N-dimethylaminostilbene)

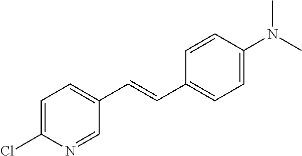 [SMILES: CN(C)c1ccc(/C=C/c2ccc(Cl)nc2)cc1]

2-Chloropyridyl-5-methylenediethylphosphonate (500 mg, 1.90 mmol) and 4-N,N-dimethylbenzaldehyde (285 mg, 1.90 mmol) were dissolved with stirring in dry dimethylformamide (5 mL). Sodium hydride (120 mg, 60% w/w, 2.0 mmol) was charged into the stirred reaction (CAUTION: gas evolution) causing an immediate colour change to deep red over the period of 2 hours. The reaction was quenched with addition of water (10 mL, cautiously), precipitating the crude product that was filtered and washed repeatedly with water to remove trace dimethylformamide. The crude yellow product was subsequently dissolved in CH₂Cl₂ (50 mL) and washed with water (3×10 mL) before organics were separated, dried over MgSO₄, filtered and removed of volatiles in vacuo to give a fine yellow solid (280 mg, 57%). ¹H NMR (500 MHz; DMSO-d₆): δ/ppm 8.51 (d, ⁴J_{HH}=2.2, 1H, PyH), 8.03 (dd, ³J_{HH}=8.4, ⁴J_{HH}=2.3, 1H, PyH), 7.44 (d, ⁴J_{HH}=8.4, 1H, PyH), 7.40 (m, AA′B′B′, 2H, ArH), 7.27 (m, AB, 1H, CH═CH), 6.97 (m, AB, 1H, CH═CH), 6.72 (m, AA′B′B′, 2H, ArH), 2.94 (s, 6H, N(CH₃)₂). ¹³C{¹H} NMR (125.7 MHz; DMSO-d₆): δ/ppm 150.3 (ArC), 147.5 (PyC), 147.4 (PyCH), 135.4 (PyCH), 133.3 (PyC), 131.8 (HC═CH), 127.9 (ArCH), 124.2 (ArC), 124.1 (PyCH), 118.2 (HC═CH), 112.1 (ArCH), 39.8 (N(CH₃)₂.

### 2-Hydrazide-pyridinyl-4-benzothiazole

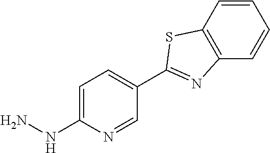 [SMILES: NNc1ccc(-c2nc3ccccc3s2)cn1]

2-Chloropyridine-4-benzothiazole (500 mg, 2.23 mmol) and hydrazine hydrate (5 mL) were refluxed in ethanol (30 mL) under nitrogen for 4 hours. A light yellow precipitate formed that on cooling to room temperature, was collected, washed with ethanol, diethylether and air dried (460 mg, 94%). ¹H NMR (500 MHz; DMSO-d₆): δ/ppm 8.69 (s, 1H, PyH), 8.28 (s, 1H, NH-Py), 8.09-8.05 (m, 2H, PyH&ArH), 7.94 (d, ³J_{HH}=7.7, 1H, ArH), 7.48 (t, ³J_{HH}=7.1, 1H, ArH), 7.36 (m, 1H, ArH), 6.86-6.84 (m, 1H, PyH), 4.39 (s, 2H, NH₂—NH). ¹³C{¹H} NMR (125.7 MHz; DMSO-d₆): δ/ppm 165.8 (BzC), 163.0 (PyCNH), 153.6 (ArC), 147.4 (PyCH), 135.5 (PyCH), 133.5 (ArC), 126.4 (ArCH), 124.6 (ArCH), 122.0 (ArCH), 121.9 (ArCH), 117.8 (PyC), 105.8 (PyCH).

### (E)-2-Hydrazide-pyridinyl-4-(4′-N,N-dimethylaminostilbene)

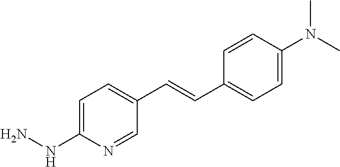 [SMILES: CN(C)c1ccc(/C=C/c2ccc(NN)nc2)cc1]

(E)-2-Chloro-pyridinyl-4-(4′-N,N-dimethylaminostilbene) (210 mg, 0.81 mmol) was refluxed in hydrazine hydrate (10 mL) under nitrogen for 16 hours. A colourless precipitate formed, that on cooling to room temperature was collected, washed repeatedly with water, followed by diethylether and air dried (200 mg, 94%). ¹H NMR (500 MHz; DMSO-d₆): δ/ppm 8.08 (bs, 1H, PyH), 7.73 (bm, 1H, PyH), 7.49 (bs, 1H, NH-Py), 7.35 (m, AA′BB′, 2H, ArH), 6.85 (aq, AB, 2H, CH═CH), 6.70 (m, AA′BB′, 2H, ArH), 4.15 (s, 2H, NH₂—NH), 2.91 (s, 6H, N(CH₃)₂). ¹³C{¹H} NMR (125.7 MHz; DMSO-d₆): δ/ppm 160.8 (PyC), 149.5 (ArC), 146.2 (PyCH), 133.1 (PyCH), 126.8 (ArCH), 125.6 (ArC), 124.8 (HC═CH), 122.8 (PyC), 121.0 (HC═CH), 112.3 (ArCH), 106.5 (PyCH), 40.0 (N(CH₃)₂).

### Diacetyl-mono-4-N,N-Dimethylaminoethyl-3-thiosemicarbazone

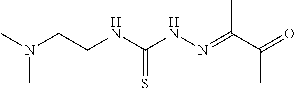

4-N,N-Dimethylaminoethyl-3-thiosemicarbazide (400 mg, 2.46 mmol) was dissolved in methanol (25 mL) and subsequently added dropwise over 1 hour to a cooled solution of 2,3-butadione (1.1 mL, 12.3 mmol) in methanol (50 mL) in the presence of catalytic HCl. The reaction was monitored by TLC (CH₂Cl₂) and on completion concentrated to dryness. The residue was extracted with CH₂Cl₂ (3×25 mL), washed with sat. sodium bicarbonate solution, before organic fractions were collated, dried over MgSO₄, and removed of volatiles. The residue was chromatographed (SiO₂, CH₂Cl₂) to give a yellow crystalline solid (420 mg, 74%). ¹H NMR (400 MHz; DMSO-d₆); δ/ppm 10.74 (s, 1H, N—NH—C═S), 8.56 (s, 1H, CH₂—NH—C═S), 3.61 (q, ³J_{HH}=5.9, 2H, CH₂), 2.46 (t, ³J_{HH}=6.5, 2H, CH₂), 2.35 (s, 3H, N═C—CH₃) 2.18 (s, 6H, N(CH₃)₂), 1.94 (s, 3H, O═C—CH₃). MS(ES⁺) m/z (calcd) 231.2018 (231.1235) {M+H⁺}.

### Diacetyl-2-(2-hydrazone-pyridinyl-4-benzothiazole)-(4-methyl-3-thiosemicarbazone) (H₂L¹)

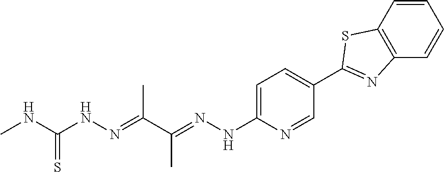 [SMILES: CNC(=S)N/N=C(C)/C(C)=N/Nc1ccc(-c2nc3ccccc3s2)cn1]

2-Hydrazinopyridine-4-benzothiazole (150 mg, 0.62 mmol) and diacetyl-mono-4-methyl-3-thiosemicarbazone (120 mg, 0.62 mmol) were refluxed in ethanol (30 mL) under nitrogen for 4 hours. A yellow precipitate formed that on cooling to room temperature, was collected, washed with ethanol then ether and air dried (195 mg, 76%). ¹H NMR, (500 MHz; DMSO-d₆): δ/ppm 10.49 (s, 1H, N—NH—C═S), 10.18 (s, 1H, N—NH—C═S), 8.88 (d, ⁴J_{HH}=2.4, 1H, PyH), 8.35-8.33 (m, 1H, CH₃—NH—C═S), 8.30 (dd, ³J_{HH}=8.8, ⁴J_{HH}=2.4, 1H, PyH), 8.11 (d, ³J_{HH}=8, 1H, ArH), 8.01 (d, ³J_{HH}=8, 1H, ArH), 7.52 (td, ³J_{HH}=8, ⁴J_{HH}=1, 1H, Ar), 7.44-7.39 (m, 2H, PyH&ArH), 3.04 (d, ³J_{HH}=4.6, 3H, NH—CH₃), 2.27 (s, 3H, N═C—CH₃), 2.25 (s, 3H, N═C—CH₃). ¹³C{¹H} NMR (125.7 MHz; DMSO-d₆): δ/ppm 178.5 (C═S), 176.7 (C═S), 154.8 (ArC), 149.6 (C═N—N), 147.7 (C═N—N), 142.8 (N═CH), 141.8 (N═CH), 139.9 (C), 130.8 (C), 130.6 (C), 127.7 (ArCH), 125.5 (ArCH), 121.4 (ArCH), 31.2 (NH—CH₃), 14.3 (Ar—CH₃), 14.2 (Ar—CH₃), 12.2 (N═C—CH₃), 11.9 (N═C—CH₃). MS(ES⁺): m/z (calcd) 398.1210 (398.1143) {M+H⁺}. HPLC Rₜ 14.56 min.

### Diacetyl-2-((E)-2-hydrazone-pyridinyl-4-(4′-N,N-dimethylaminostilbene))-(4-methyl-3-thiosemicarbazone) (H₂L²)

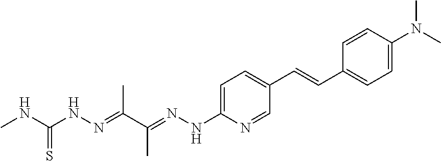

(E)-2-Hydrazino-pyridinyl-4-(4′-N,N-dimethylaminostilbene) (50 mg, 0.20 mmol) and diacetyl-mono-4-methyl-3-thiosemicarbazone (35 mg, 0.20 mmol) were refluxed in ethanol (30 mL) under nitrogen for 4 hours. A yellow precipitate formed that on cooling to room temperature, was collected, washed with ethanol then ether and air dried. ¹H NMR (500 MHz; DMSO-d₆): δ/ppm 10.12 (s, 1H, N—NH—C═S), 9.98 (s, 1H, N—NH-Py), 8.31 (m, 1H, CH₃—NH—C═S), 8.29 (d, ⁴J_{HH}=2.2, 1H, PyH), 7.82 (dd, ³J_{HH}=8.8, ⁴J_{HH}=2.3, 1H, PyH), 7.40 (m, AA′BB′, 2H, ArH), 7.25 (d, ³J_{HH}=8.7, 1H, PyH), 7.04 (m, AB, 1H, CH═CH), 6.92 (m, AB, 1H, CH═CH), 6.72 (m, AA′BB′, 2H, ArH), 3.04 (d, 3H, ³J_{HH}=4.6, NH—CH₃), 2.92 (s, 6H, N(CH₃)₂), 2.23 (s, 3H, N═C—CH₃), 2.22 (s, 3H, N═C—CH₃). ¹³C {¹H} NMR (125.7 MHz; DMSO-d₆): δ/ppm 183.6 (C═S), 160.9 (PyC), 155.0 (ArC), 153.9 (C═N—N), 151.2 (PyCH), 149.4 (C═N—N), 139.5 (PyCH), 132.4 (ArCH), 132.1 (HC═CH), 131.4 (PyC), 130.5 (ArC), 125.5 (HC═CH), 117.5 (ArCH), 112.3 (PyCH), 45.2 (N(CH₃)₂), 36.3 (NH—CH₃), 16.6 (N═C—CH₃), 16.1, (N═C—CH₃). HPLC Rₜ 11.43 min.

### Diacetyl-2-((E)-2-hydrazino-pyridinyl-4-(4′-N,N-dimethylaminostilbene))-(4-dimethylaminoethyl-3-thiosemicarbazone) (H₂L³)

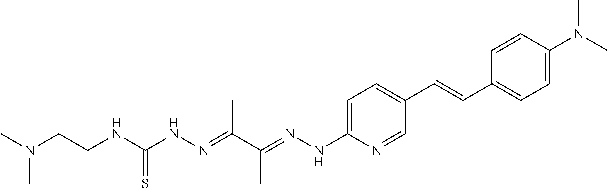

(E)-2-Hydrazino-pyridinyl-4-(4′-N,N-dimethylaminostilbene) (100 mg, 0.39 mmol) and diacetyl-mono-4-dimethylaminoethyl-3-thiosemicarbazone (110 mg, 0.47 mmol) were refluxed in ethanol (30 mL) under nitrogen for 4 hours in the presence of catalytic cone. HCl. The reaction was followed by TLC (EtOAc), and on completion allowed to cool to room temperature before filtration through celite. The filtrate was concentrated to 5 mL before trituration with diethyl ether precipitated a crystalline yellow solid. The precipitate was collected, washed with ether and air dried (80 mg, 44%). Elem anal Found (calcd) for C₂₁H₂₇S: C, 61.40 (61.77); H, 6.75 (7.34); N, 23.85 (24.01). ¹H NMR (500 MHz; DMSO-d₆): δ/ppm 10.47 (s, 1H, N—NH—C═S), 10.04 (s, 1H, N—NH-Py), 9.95 (bs, 1H, [C—NH(CH₃)₂]⁺), 8.47 (m, 1H, CH₂—NH—C═S), 8.28 (d, ⁴J_{HH}=2.2, 1H PyH), 7.90 (m, 1H, PyH), 7.38 (m, AA′BB′, 2H, ArH), 7.24 (d, ³J_{HH}=8.8, 1H, PyH), 7.03 (m, AB, 1H, CH═CH), 6.91 (m, AB, 1H, CH═CH), 6.70 (m, AA′BB′, 2H, ArH), 3.95 (m, 2H, N—CH₂), 2.91 (s, 6H, N(CH₃)₂), 2.81 (bs, 6H, N(CH₃)₂), 2.24 (s, 3H, N═C—CH₃), 2.22 (s, 3H, N═C—CH₃). ¹³C{¹H} NMR (125.7 MHz; DMSO-d₆): δ/ppm 183.6 (C═S), 160.9 (PyC), 155.0 (ArC), 153.9 (C═N—N), 151.2 (PyCH), 149.4 (C═N—N), 139.5 (PyCH), 132.4 (ArCH), 132.1 (HC═CH), 131.4 (PyC), 130.5 (ArC), 125.5 (HC═CH), 117.5 (ArCH), 112.3 (PyCH), 45.2 (N(CH₃)₂), 36.3 (NH—CH₃). 16.6 (N═C—CH₃), 16.1 (N═C—CH₃). MS (ES*) m/z (calcd) 467.27 (467.2627) {M+H⁺}. HPLC Rₜ 9.24 min.

### Example 1

### Diacetyl-2-(2-hydrazonato-pyridinyl-4-benzothiazole)-(4-methyl-3-thiosemicarbazonato)copper-(II)

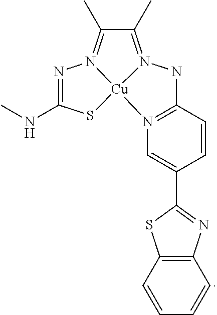

H₂L¹ (50 mg, 0.12 mmol) and copper(II) acetate (27 mg, 0.13 mmol) were refluxed in ethanol (10 mL) under nitrogen for 2 hours. A dark purple precipitate formed that on cooling to room temperature, was collected, washed with ethanol (3×5 mL), and air dried (15 mg, 27%). MS(ES⁺): m/z (calcd) 459.0166 (459.0283) {M+H⁺}. HPLC:Rₜ 13.49 min. Crystals suitable for single-crystal X-ray diffraction were grown from slow diffusion of diethylether at room temperature into a degassed solution of H₂L¹ and copper(I) tetrafluoroborate in dimethylformamide.

### Example 2

### Diacetyl-2-((E)-2-hydrazonato-pyridinyl-4-(4′-N,N-dimethylaminostilbene))-(4-methyl-3-thiosemicarbazonato) copper(II)

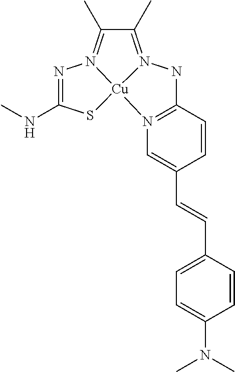

H₂L² (50 mg, 0.12 mmol) was dissolved in ethanol (10 ml) heated to reflux and subsequently treated with copper(II) acetate (27 mg, 0.13 mmol). The reaction darkened immediately affording a deep blue solution that was stirred for 2 hours. On cooling to room temperature the reaction was concentrated and chromatographed, (gradient 2% MeOH/CH₂Cl₂). Fractions of a deep blue colour were collated and removal of volatiles gave a near black solid (35 mg, 61%). MS(ES⁺): m/z (calcd) 471.1264 (471.1188) {M+H⁺}. HPLC Rₜ 11.79 min. Crystals suitable for single-crystal X-ray diffraction were grown from slow diffusion of diethylether at room temperature into a degassed solution of H₂L² and copper(I) tetrafluoroborate in dimethylformamide.

### Example 3

### Diacetyl-2-((E)-2-hydrazino-pyridinyl-4-(4′N,N-dimethylaminostilbene))-(4-dimethylaminoethyl-3-thiosemicarbazonato)copper(II)

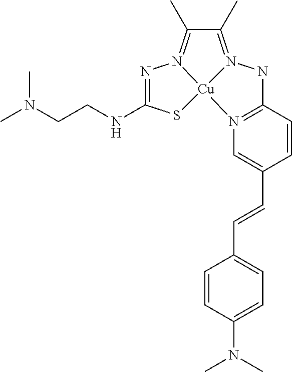

H₂L³ (20 mg, 0.04 mmol) was suspended in DCM (10 mL) and heated to near reflux. Copper acetate (10 mg, 0.05 mmol) was added to the reaction causing a gradual solution colour change to near black. The reaction was refluxed for 2 hours with monitoring by TLC (10% MeOH/DCM/0.1% NEt₃). The reaction was removed from heat and concentrated to dryness. The residue was purified by flash chromatography eluting deep blue fractions (12 mg, 54%). Elem anal Found (calcd) for C₂₁H₂₅CuN₇S: C, 55.47 (54.58); H, 4.66 (6.11); N, 21.64 (21.22). MS(ES⁺) m/z (calcd) 528.18 (528.1767) {M+H⁺}. HPLC Rₜ 9.35 min.

### Biological Data

The Interaction of Cu^{II}L¹ and Cu^{II}L² with Aβ Plaques in Human Brain Tissue

The potential of Cu^{II}L¹ and Cu^{II}L² to bind Aβ plaques was investigated in serial sections of post-mortem brains of AD subjects as well as age-matched controls. Human brain tissue (7 μm-serial sections) was pre-treated with BSA to prevent non-selective binding and then treated with solutions of Cu^{II}L¹ and Cu^{II}L² (150 μm in 15% DMSO/PB, 20 μM, pH 7.4).

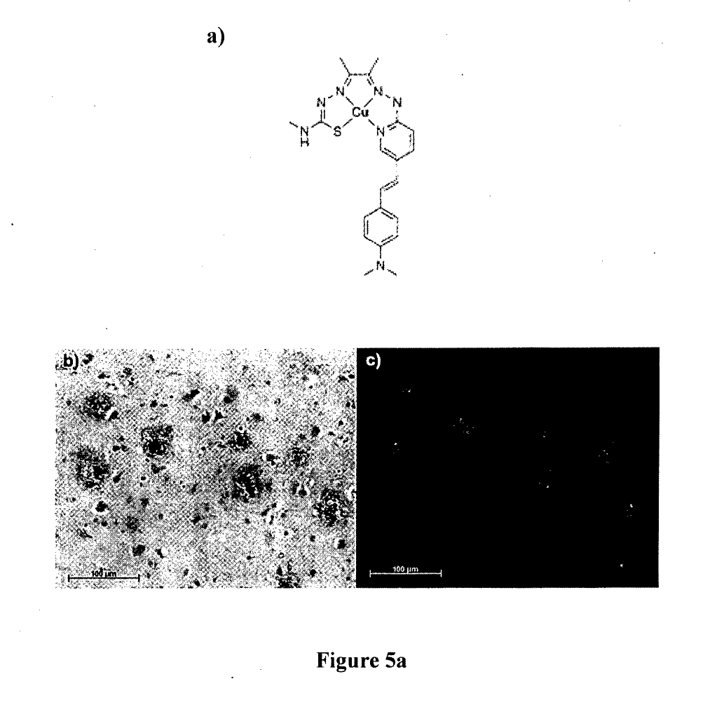

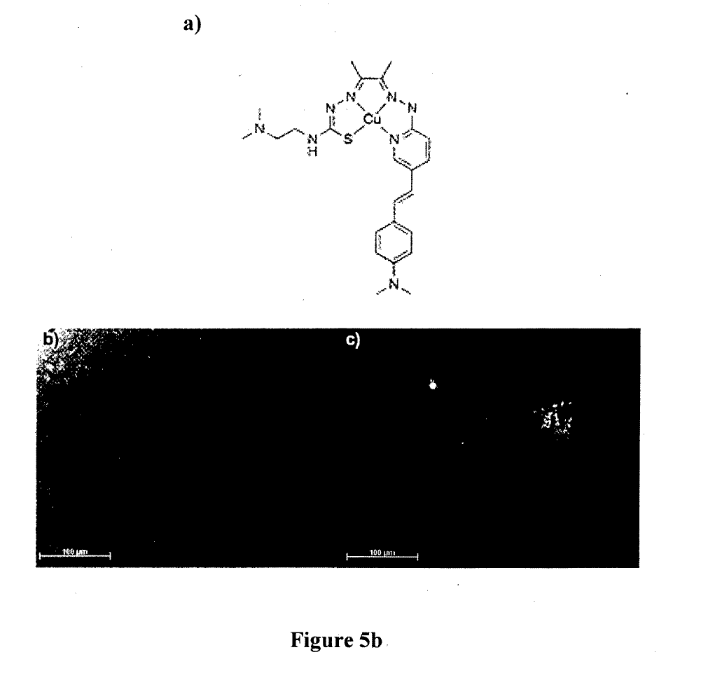

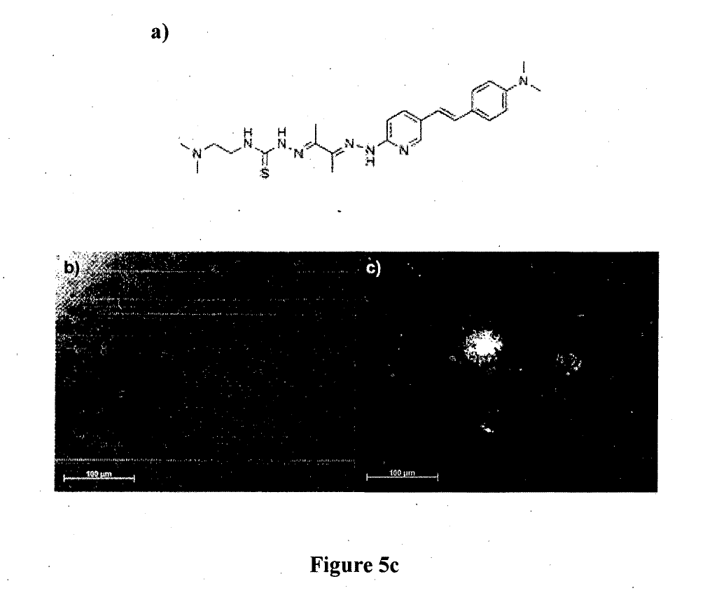

The tissue was subsequently examined by fluorescent microscopy (epi-fluorescence, λₑₓ=420 nm, λₑₓ=470 nm) and compared to a sequential brain tissue cross section immunostained with an Aβ antibody (1 E8). As Aβ plaques are typically between 40-60 μm so consecutive 7 μm-serial sections often contain the same Aβ plaque, (¹⁰⁾ therefore co-localisation between the immuno-stained and epi-fluorescence images indicates whether the compound binds to Aβ plaques. Cu^{II}L¹ failed to bind to Aβ plaques, as there was no observed co-localised epi-fluorescence. However, as evident in FIG. 5b and FIG. 5d, co-localisation of the immuno-stained and epi-fluorescence images clearly demonstrates that Cu^{II}L² binds selectively to Aβ plaques in a manner that reveals with exquisite detail the filamentous nature of the extracellular aggregates.

Radiolabelling with ⁶⁴Cu and Biodistribution in Mice

TABLE 1
Partition coefficients for ⁶⁴Cu complexes

| Ligand | Log D of complex |
|---|---|
| atsmH2a | 1.48 |
| ThypyH2 | 1.26 |
| ThynicH2 | −1.43 |
| H2L2 | 1.46 |
| H2L3 | 1.52 |

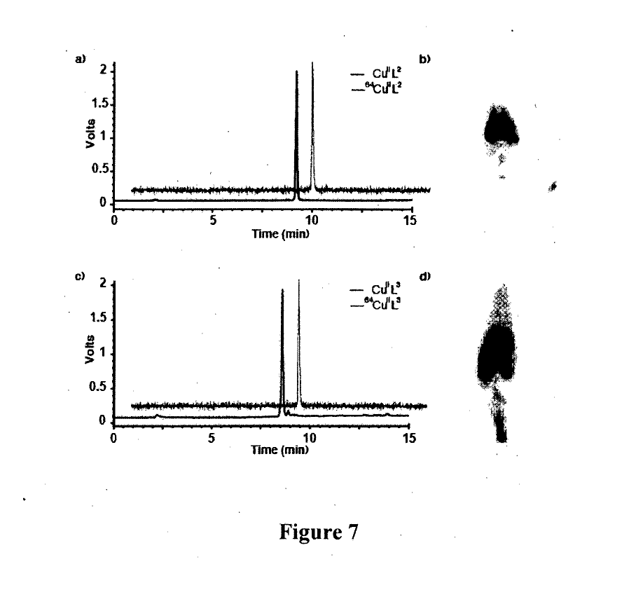

Radiolabelled ⁶⁴Cu^{II}L² was prepared in >90% radiochemical purity according to radio-HPLC by the coordination of H₂L² with ⁶⁴Cu^{II} at room temperature in PBS buffer (0.01 M) at pH 7.4. The identity of the radiolabelled product was confirmed by a comparison with the non-radioactive analogue Cu^{II}L² (FIG. 7). Preliminary small animal PET studies were undertaken ni Balb/c mice (FIG. 7b), where following intravenous tail vein injection of approximately 13 MBq of ⁶⁴Cu^{II}L², uptake throughout the body was imaged 5 minutes post-injection.

TABLE 2
Biodistribution of radioactivity after
injection of ⁶⁴Cu¹¹L³ in Balb/c mice

Time after injection (min)

| Tissue | 2 | 30 |
|---|---|---|
| Blood | 4.25(0.56) | 1.70(0.29) |
| Lungs | 42.16(20.27) | 17.74(4.55) |
| Heart | 12.08(1.50) | 5.26(0.86) |
| Liver | 11.28(5.02) | 7.38(0.59) |
| Kidneys | 15.25(2.02) | 5.17(0.63) |
| Muscle | 0.51(0.50) | 0.90(0.13) |
| Spleen | 18.02(3.35) | 8.24(0.89) |
| Brain | 1.11(0.20) | 0.38(0.09) |

Each value represents the mean (SD) for three animals expressed as % injected dose per organ.

### REFERENCES

(1) Cowley, A.; Dilworth, J.; Donnelly, P.; White, J. Inorg. Chem. 2006, 45, 496-498.
(2) Cowley, A. R.; Dilworth, J. R.; Donnelly, P. S.; Labisbal, E.; Sousa, A. J. Am. Chem. Soc. 2002, 124, 5270-5271.
(3) CrysAlis CCD 2007.
(4) Sheldrick, G. SHELX97 [Includes SHELXS97, SHELXL97]—Programs for Crystal Structure Analysis 1998.
(5) Farrugia, L. J. Journal of Applied Crystallography 1999, 32, 837-838.
(6) Johnson, C.; Burnett, M. ORTEP-3 for Windows 1998, 128.
(7) Paterson, B. M.; Karas, J. A.; Scanlon, D. B.; White, J. M.; Donnelly, P. S. Inorg. Chem. 2010, 49, 1884-1893.
(8) Cowley, A. R.; Dilworth, J. P.; Donnelly, P. S.; Heslop, J. M.; Ratcliffe, S. J. Dalton Trans. 2007, 209-217.
(9) Gottlieb, H.; Kotlyar, V.; Nudelman, A. J Org Chem 1997, 62, 7512-7515.
(10) Fodero-Tavoletti, M. T.; Smith, D. P.; McLean, C. A.; Adlard, P. A.; Barnham, K. J.; Foster, L. E.; Leone, L.; Perez, K.; Cortes, M.; Culvenor, J. G.; Li, Q.-X.; Laughton, K. M.; Rowe, C. C.; Masters, C. L.; Cappai, R.; Villemagne, V. L. J Neurosci 2007, 27, 10365-10371.

1. A metal complex of formula (I) or a salt thereof: 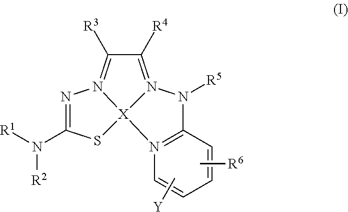
wherein:
X is Cu, Ga or Tc═O
Y is 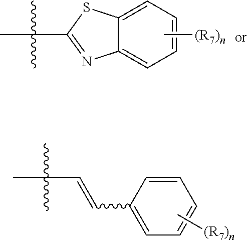
R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocycyl;
R³ and R⁴ are independently selected from hydrogen or C₁-C₄ alkyl, or R³ and R⁴ together form an optionally substituted aryl or optionally substituted cycloalkyl group;
R⁵ is selected from hydrogen or C₁-C₄ alkyl;
R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy, or amino;
R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy, or amino; and
n is 0-4.

2. A metal complex according to claim 1 of formula (Ia) or formula (Ib) or a salt thereof: 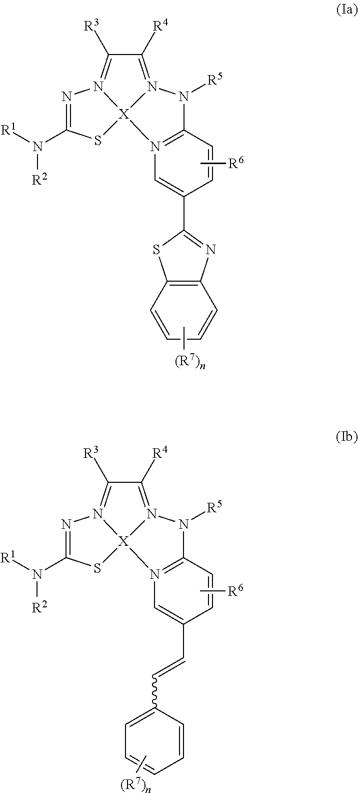
wherein:
X is Cu, Ga or Tc═O;
R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;
R³ and R⁴ are independently selected from hydrogen or C₁-C₄ alkyl, or R³ and R⁴ together form an optionally substituted aryl or optionally substituted cycloalkyl group;
R⁵ is selected from hydrogen or C₁-C₄ alkyl;
R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy, or amino;
R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy, or amino; and
n is 0-4.

3. A metal complex according to claim 2 wherein the complex is a metal complex of formula (Ia), or a salt thereof.

4. A metal complex according to claim 2 wherein the complex is a metal complex of formula (Ib), or a salt thereof.

5. A metal complex according to any one of claims 1 to 4 wherein R¹ is hydrogen.

6. A metal complex according to any one of claims 1 to 4 wherein R¹ is hydrogen and R² is an optionally substituted C₁-C₃ alkyl.

7. A metal complex according to any one of claims 1 to 4 wherein R¹ is hydrogen and R² is C₁-C₃ alkyl.

8. A metal complex according to any one of claims 1 to 4 wherein R¹ is hydrogen and R² is a substituted C₁-C₃ alkyl.

9. A metal complex according to any one of claims 1 to 4 wherein R¹ is hydrogen and R² is a terminally substituted C₁-C₃ alkyl.

10. A metal complex according to any one of claims 1 to 4 wherein R¹ is hydrogen and R² is a C₁-C₃ alkyl terminally substituted with a group selected from halogen, amino, C₁-C₃ dialkyl amino, C₁-C₃ monoalkyl amino, aryl, carboxyl, trihalomethyl, acyl, and N-containing heteroaryl or N-containing heterocyclyl.

11. A metal complex according to any one of claims 1 to 4 wherein R² is a C₁-C₃ alkyl terminally substituted with C₁-C₃ dialkyl amino and C₁-C₃ monoalkyl amino, or a bioisostere thereof.

12. A metal complex according to any one of claims 1 to 4 wherein R¹ is hydrogen and R² is C₁-C₃ alkyl or di C₁-C₃ alkyl amino ethyl.

13. A metal complex according to any one of claims 1 to 4 wherein R¹ is hydrogen and R² is methyl or dimethylaminoethyl.

14. A metal complex according to any one of claims 1 to 4 wherein R³ and R⁴ are independently C₁-C₃ alkyl.

15. A metal complex according to any one of claims 1 to 4 wherein R³ and R⁴ are both methyl.

16. A metal complex according to any one of claims 1 to 4 wherein R¹ is hydrogen, and R²-R⁴ are independently C₁-C₃ alkyl.

17. A metal complex according to any one of claims 1 to 4 wherein R¹ is hydrogen, R³ and R⁴ are C₁-C₃ alkyl and R² is dimethylaminoethyl or a bioisostere thereof.

18. A metal complex according to any one of claims 1 to 4 wherein R⁵ is hydrogen.

19. A metal complex according to any one of claims 1 to 4 wherein R¹ and R⁵ are hydrogen, and R²-R⁴ are independently C₁-C₃ alkyl.

20. A metal complex according to any one of claims 1 to 4 wherein R¹ and R⁵ are hydrogen, R³ and R⁴ are C₁-C₃ alkyl and R² is dimethylaminoethyl or a bioisostere thereof.

21. A metal complex according to any one of claims 1 to 4 wherein R⁶ is hydrogen.

22. A metal complex according to any one of claims 1 to 4 wherein R⁶ is hydrogen and n=0.

23. A metal complex according to any one of claims 1 to 4 wherein R⁶ is hydrogen and n=1.

24. A metal complex according to any one of claims 1 to 4 wherein R¹, R⁵, and R⁶ are hydrogen, R³-R⁴ are independently C₁-C₃ alkyl or together form an optionally substituted aryl or optionally substituted cycloalkyl group and n=0, or 1.

25. A metal complex according to any one of claims 1 to 4 wherein R⁷ is dimethylamino.

26. A metal complex according to any one of claims 1 to 25 wherein X is Cu.

27. A metal complex according to claim 26 wherein X is ⁶⁴Cu.

28. A metal complex according to any one of claims 1 to 25 wherein X is Ga, preferably ⁶⁸Ga.

29. A metal complex according to claim 27 or 28 for use in PET imaging.

30. A method of diagnosing an amyloid disorder comprising:
(i) administering a detectable quantity of a complex according to any one of claims 1 to 29 or a salt thereof to a patient, and
(ii) detecting the binding of the complex to an amyloid deposit in said patient.

31. A compound of formula (IIa) or a salt thereof: 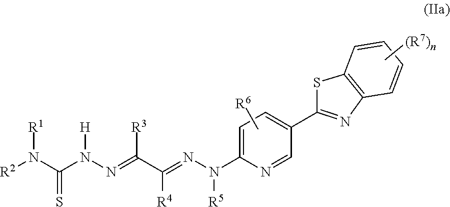
wherein
R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;
R³ and R⁴ are independently selected from hydrogen or C₁-C₄ alkyl, or R³ and R⁴ together form an optionally substituted aryl or optionally substituted cycloalkyl group;
R⁵ is selected from hydrogen or C₁-C₄ alkyl;
R⁶ is selected from hydrogen, hydroxy, halogen, carboxy, optionally substituted C₁-C₄ alkyl, optionally substituted C₁-C₄ alkoxy, optionally substituted aryl, optionally substituted aryloxy, or amino;
R⁷ at each occurrence is independently selected from hydroxy, halogen, carboxy, optionally substituted C₁-C₄ alkyl, optionally substituted C₁-C₄ alkoxy, optionally substituted aryl, optionally substituted aryloxy, or amino; and
n is 0-4.

32. A compound of formula (IIb) or salts thereof: 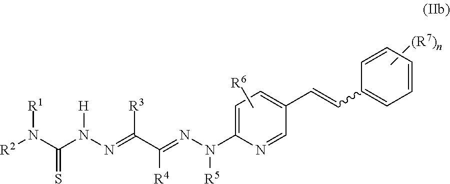
wherein:
R¹ and R² are independently selected from hydrogen, optionally substituted C₁-C₆ alkyl, amino, —N═R⁸ (when R⁸ is optionally substituted alkyl or optionally substituted aryl), optionally substituted aryl, optionally substituted heteroaryl or optionally substituted heterocyclyl;
R³ and R⁴ are independently selected from hydrogen or C₁-C₄ alkyl, or R¹ and R⁴ together form an optionally substituted aryl or optionally substituted cycloalkyl group;
R⁵ is selected from hydrogen or C₁-C₄ alkyl;
R⁶ is selected from hydrogen, hydroxy, halogen, carboxy, acyl, optionally substituted C₁-C₄ alkyl, optionally substituted C₁-C₄ alkoxy, optionally substituted aryl, optionally substituted aryloxy or amino;
R⁷, at each occurrence, is independently selected from hydroxy, halogen, carboxy, optionally substituted C₁-C₄ alkyl, optionally substituted C₁-C₄ alkoxy, optionally substituted aryl, optionally substituted aryloxy or amino; and
n is 0-4.
